(19) United States
(12) Patent Application Publication
(10) Pub. No.: US 2008/0131891 A1
(43) Pub. Date: Jun. 5, 2008

(54) Biochemical Reagents And Their Uses

(75) Inventors: David James Squirrell, Wiltshire (GB); Martin Alan Lee, Wiltshire (GB)

Correspondence Address: POLSINELLI SHALTON FLANIGAN SUELTHAUS PC, 700 W. 47TH STREET, SUITE 1000, KANSAS CITY, MO, 64112-1802, US

(73) Assignee: ENIGMA DIAGNOSTICS LTD, Wiltshire, GB (GB)

(21) Appl. No.: 11/814,849

(22) PCT Filed: Feb. 1, 2006
(86) PCT No.: PCT/GB06/00350

§ 371 Date: Sep. 27, 2007

(30) Foreign Application Priority Data
Feb. 1, 2005 (GB) 0502010.2

(51) Int. Cl. C12Q 1/68; C07H 21/04; C12P 19/34

(52) U.S. Cl. 435/6; 536/23.1; 435/91.2; 536/24.31; 536/24.33

(57) ABSTRACT

A method for adding a first and a second functional nucleic acid sequence to a reaction mixture, in particular an amplification reaction mixture in a predetermined stoichiometry and/or at a predetermined point in time, said method comprising adding to the reaction mixture an oligonucleotide comprising a first and a second functional nucleic acid sequence separated by a spacer sequence, said spacer sequence comprising a region which, when double stranded, comprises a cleavable region, forming a cleavable double stranded region within the spacer region of said oligonucleotide, and cleaving the double stranded region within said oligonucleotide. Oligonucleotides for use in the method, and comprising a first and a second functional nucleic acid sequence, such as primers or probes used in an amplification reaction, separated by a spacer sequence, is also provided.

The present invention relates to biochemical reagents, in particular oligonucleotides, and their uses, in particular in amplification reactions, such as the polymerase chain reaction.

It is well known that the polymerase chain reaction (PCR) can suffer from spurious artefacts caused by non-specific primer-template and primer-primer interactions. The latter may seriously lower the limit of detection, such products competing for reaction components that are rapidly depleted towards reaction completion. The interaction between forward and reverse primers prior to the first denaturation step is the primary, but not exclusive, cause of problems.

A variety of approaches have been taken to address this problem, and these have become known as “hot start” options. Generally, these focus on controlling the activity of the polymerase enzyme until the desired reaction point, specifically a particular temperature is reached in the reaction vessel.

For instance, the activity of the enzyme may be inhibited at lower temperatures by physical separation (wax), sequestration using for example antibodies which bind the polymerase such as anti-Taq antibodies, or chemical modification (acitonate for TaqGold). Enzymatic inhibition by addition of inhibitory amounts of pyrophosphate, which are removable using a suitable pyrophosphatase enzyme has also been proposed (WO02/088387).

Another method involves the use of hotstart polymerase enzymes, the most effective of which require heat activation for considerable time. These are, however, less than favourable on fast machines that can carry out the overall PCR process in less time than that required for activation.

Automated synthesis of oligonucleotide primers is now routine. The forward and reverse primers are synthesised in separate operations. They are often required in the reaction at the same concentration. Obtaining the correct reaction stoichiometry of each primer requires batch-to-batch empirical determination because there are generally no quick, accurate and precise methods for routinely determining oligonucleotide yield.

The applicants have developed a new approach to these problems.

According to the present invention there is provided a method for adding a first and a second functional nucleic acid sequence to a reaction mixture, said method comprising adding to the reaction mixture an olignonucleotide as described above, forming a cleavable double stranded region within the spacer region of said oligonucleotide, and cleaving the double stranded region within said olignonucleotide.

Using oligonucleotides of this type, the method allows for adding a first and a second functional nucleic acid sequence to a reaction mixture in a predetermined stoichiometry and/or at a predetermined point in time. The stoichiometry is fixed by the ratio of the sequences contained in the oligonucleotide, so that in an oligonucleotide containing a single copy of a first functional nucleic acid and a single copy of a second functional nucleic acid, the ratio of the amount of the first to the second functional nucleic acid will be 1:1.

Furthermore, the point at which individual functional nucleic acids become available may be controllable to the point at which the cleaving reaction occurs. This may be controlled by for example adding or activating a cleaving agent such as a restriction enzyme at the particular point in time at which release of functional nucleic acid sequences is required.

Oligonucleotides, in particular those which are suitable for carrying out the methods described herein form a further aspect of the invention.

According to a further aspect of the present invention, there is provided an oligonucleotide comprising a first and a second functional nucleic acid sequence separated by a spacer sequence, said spacer sequence comprising a region which, when double stranded, comprises a cleavable region. The oligonucleotide is suitably designed to be suitable for a method as described herein.

Double stranded regions may be formed in various ways, for example, by addition of a small DNA or RNA oligonucleotide, which is able to hybridise to the said region of the spacer sequence. Thereafter the thus formed double stranded region is cleavable, for example using a restriction endonuclease or an RNAseH or enzyme having RNAseH activity such as some reverse transciptase (RT) enzymes which cleaves or digests the double stranded region. A combination of a first oligonucleotide as described above, and a second oligonucleotide which is shorter than said first oligonucleotide and which is capable of hybridising to said first oligonucleotide to produce said double stranded cleavable region, forms a further aspect of the invention.

Preferably however, the spacer sequence of the oligonucleotide includes two complementary regions which can hybridise together to form a double stranded cleavable region.

By “functional nucleic acid sequence” is meant that the nucleic acid sequence has an independent function, for example it has a biological activity, or more usually, is may be useful in an assay or reaction, for example by acting as an amplification primer or probe.

Such oligonucleotides are useful in that they are multifunctional in nature and may be used to provide separate functional sequences in stoichiometric amounts for use, for example in assays or the like.

Such an oligonucleotide is particularly useful in the context of an amplification reaction such as a PCR reaction, since the problems with the stoichiometry of the primer mix is effectively eliminated.

Thus in a preferred embodiment, the first and second nucleic acid sequences comprise a first and second primer sequence. However, other sequences may be provided, for example, one or both of the said first and second sequences may comprise optionally labelled probe sequences, which are used in an assay, and which may be generated in situ.

The 5′ end of any primer confers little if any specificity on a priming event. In fact long tails of non-specific sequence are often exploited in cloning and generic probing methods. Therefore, it is not essential that the oligonucleotide is cleaved precisely at the 5′ end of the desired first and second primer sequences. The fact that some of the spacer region may be fused to the 5′ end of the formed first or second primer will not affect their utility in an amplification reaction.

In addition, the oligonucleotide may be designed to be cleaved under particular conditions, for example of elevated temperature, giving the possibility that this can be used to implement a form of “hotstart” PCR. If necessary, a preliminary high temperature incubation step is carried out for a sufficient period of time to ensure that adequate amounts of the oligonucleotide is cleaved, for example, cleavage of the oligonucleotide should be near completion.

In effect, the primers used in the amplification reaction are cloaked until the reaction is started.

Many miss-priming events occur when the sample and the reaction mixture is initially being heated. High stringency is only achieved at high temperatures. Therefore during the initial heating phase, in which the temperature is typically increased from 0° C. to 95° C. during the initial melt phase, it is possible that primers present will anneal to each other or non-specifically to the template.

However, by using the oligonucleotide of the invention, it is possible to ensure that one or both the primers are not present in a free state until above such annealing temperatures, and so the possibility of miss-priming is substantially reduced.

Ideally the cleavage reaction which leads to the formation of the free primer sequences, takes place at or around the denaturing temperature of the nucleic acid. This means that the second primer in particular, where it is present in the oligonucleotide in a 3′-5′ orientation (as discussed in more detail below), only be formed at a temperature higher than it can significantly interact with the template and undergo primer extension.

In a particular embodiment, the double stranded cleavable region is cleavable using an enzyme.

Restriction enzymes, such as restriction endonucleases, which can effectively cut dsDNA including DNA formed from hairpin structures are well known. The cutting frequencies vary and some have rare target sequences, for example of about 7-8 base pairs in length, making them useful in particular where long template DNA is being investigated. It is necessary to ensure however that the sequence cleaved is not a native sequence of the target area of the template being used in the amplification, so that the reaction is not compromised.

Heat active, and preferably thermostable restriction enzymes are also available that can facilitate high temperature cleavage.

Particular examples of such enzymes include Bsm 1 from Bacillus stearothermophilus, BstEII from Bacillus stearothermophilus ET, and Not 1 from Nocardia otidiscaviarum which recognise rare sequences, and Taq 1 from Thermus aquaticus, which is more thermostable. However, enzymes with the particularly desired properties may be engineered or isolated from suitable sources. For instance, restriction enzymes which are substantially active only at high or elevated temperatures, such as those encountered during the denaturation stage of an amplification reaction such as PCR may be isolated from thermophilic organisms such as thermophilic archeons, and in particular hyperthermophilic archeons. Examples of thermophilic organisms which may be the source of suitable restriction enzymes, (as well as other enzymes which may be utilised, for example in an amplification reaction such as PCR) include Thermus aquaticus, Thermus thermophilus, Thermus species NH, Thermus brockianus, Pyrococcus furiosus, Thermococcus litoralis, Sulfolbus acidicaldarius, Thermococcus litoralis or Aeropyrum pernix.

However, many other restriction enzymes may be utilised, in particular where they are used in a way in which they are not expected to withstand or be active at high temperatures. For instance, in many cases, it may be appropriate to add the restriction enzyme to the reaction mixture shortly before the start of the reaction and allow it to cleave the olignucleotide to release the functional nucleic acid sequences. In this case, inactivation of the restriction enzyme as a result of heating will not impact upon the success of the procedure, as the functional nucleic acid sequences have then been released.

In order to use such enzymes, it is necessary only to engineer the sequence recognised by them into the spacer region of the oligonucleotide. Examples of these particular sequences and the enzymes which cleave these are set out in the following

TABLE

| Enzyme | Target sequence |
|---|---|
| Bsm 1 from Bacillus | 5′ GAATGCN/3′ 3′ CTTAC/GN5′ |
| stearothermophilus | |
| BstEII from Bacillus | 5′ G/gtnacc 3′ |
| stearothermophilus ET, | |
| Not 1 from Nocardia | 5′ gc/ggccgc-3′ |
| otidiscaviarum | |
| Taq 1 from Thermus aquaticus | 5′ t/cga3′ |

where N is any base.

Other enzymes may cut nucleic acid hybrids including, for example, RNAse H which will cleave an RNA portion of an RNA-DNA duplex. Thus such enzymes can be used to cleave an oligonucleotide as described above, which comprises a DNA-RNA copolymer, and wherein the said spacer sequence forms an RNA/DNA duplex, either with an added small oligonucleotide or with a complementary region found within the spacer sequence.

Additionally, there are reports of self-cleaving nucleic acid sequences such as ribozymes and unstable DNA nucleotide structures that appear to self-cleave (K. K. Singh et al., Ribozymes and siRNA protocols, Second Edition, Mar. 2004, pps. 033-048, ISBN: 1-59259-746-7, Series: Methods in Molecular Biology, Volume #: 252, and Carmi N. et al.: Chem Biol. 1996 December; 3(12):1039-46. These may also be used, in particular in oligonucleotides which include complementary regions able to form hairpin structures.

Synthesis of oligonucleotides is normally carried out 3′ to 5′. However, it is also possible to carry out synthesis 5′ to 3′ using specialised phosphoramadite monomers provided the first base is a 3′ base. It is also possible to switch directions during synthesis. With such a chemistry it is possible to carry out a back-to-back synthesis such that both ends of the oligonucleotide are effectively 5′ ends. The single 3′ base, followed by a 5′-3′ stretch of bases, for instance from 20-30 bases, will not interfere in the ability of the sequence to act as a primer sequence.

Thus, in a particular embodiment, one of the sequences is orientated as a 5′-3′ sequence, and wherein the other sequence is orientated 3′-5′ within the single oligonucleotide. These are arranged so that both ends of the oligonucleotide are 5′ ends. This has the advantage of ensuring that, where these sequences are first and second primer sequences, neither primer sequence can give rise to premature priming events, as there are no “free” 3′ ends, prior to cleavage.

In a further particular embodiment, where there are two complementary regions of the spacer sequence within the oligonucleotide are spaced from each other. This provides more flexibility in the structure, and so ensures that the complementary regions can, in the normal course, hybridise together easily, to form a “hairpin” type structure.

Furthermore, the space between the two complementary regions can comprise a third or additional functional nucleic acid sequence, which may be useful in a particular assay.

However, it is also possible to include additional functional sequences in oligonucleotides which do not include complementary regions, provided in that case, the spacer region includes sufficient regions which are able to form complementary regions to allow them to cleave the oligonucleotide a sufficient number of times to release all the functional sequences therein.

Similarly, further sequences may be “carried” within the space between the two complementary regions if desired, provided that, if they are intended for use separately, they are separated by regions which are complementary to other regions within the oligonucleotide and wherein any thus formed hybrids are cleavable as described above.

Particular examples of such additional functional sequences include, for example probe sequences. These may be particularly useful when the first and second sequences are first and second primer sequences, as the cleavage of the oligonucleotide will give rise to a pair of primers and a probe, which are the basic components of many amplification assays, in particular those able to conduct real-time monitoring of amplification, such as the well-known “TaqMan™” method, as well as methods described for example in WO 99/28500.

However, this combination of reagents may also be generated where the first and second sequences comprise a first primer and a probe, and where the said third sequence is a second primer.

In order to release the third sequence (as well as any further sequences), from both the first and second sequences, it may be necessary to ensure that the cleavable region, and/or the means used to effect the cleavage, are suited to cut both strands of the duplex. Thus, for example, where RNase H is used as the cleavage means, it will be necessary to ensure that the oligonucleotide is an RNA/DNA copolymer which includes an appropriate number of separate RNA regions to ensure that complete cleavage is effected. For example, where there is third sequence only in the space between the complementary regions of the olignucleotide, it will be necessary to ensure that there are two RNA regions within the oligonucleotide, one arranged to release the first sequence from the oligonucleotide, and one arranged to release the second sequence.

Alternatively, the spacer between the functional sequences, in particular between complementary regions where present, may comprise a “self-cleaving” functionality such as a ribozyme, which will be effective to cleave the first and second sequences at the appropriate time.

Any of the first, second or third functional nucleic acid sequences may carry one or more labels as required. In particular, where these are primer or probe sequences, they may be detectably labelled, preferably in such as way as to give rise to the possibility of detecting amplification product in a homogenous manner, especially in “real-time”. In particular, it may be advantageous to detectably label any probe sequences within the oligonucleotide.

Such labels include visible labels and in particular fluorescent labels. In particular one or more fluorescent labels may be arranged to undergo fluorescent energy transfer (FET) and particularly fluorescent resonant energy transfer (FRET) during an assay, and therefore these labels may be included in the oligonucleotides described above.

There are two commonly used types of FET or FRET probes, those using hydrolysis of nucleic acid probes to separate donor from acceptor, and those using hybridisation to alter the spatial relationship of donor and acceptor molecules.

Hydrolysis probes are commercially available as TaqMan™ probes. These consist of DNA oligonucleotides that are labelled with donor and acceptor molecules. The probes are designed to bind to a specific region on one strand of a PCR product. Following annealing of the PCR primer to this strand, Taq enzyme extends the DNA with 5′ to 3′ polymerase activity. Taq enzyme also exhibits 5′ to 3′ exonuclease activity. TaqMan™ probes are protected at the 3′ end by phosphorylation to prevent them from priming Taq extension. If the TaqMan™ probe is hybridised to the product strand, an extending Taq molecule may also hydrolyse the probe, liberating the donor from acceptor as the basis of detection. The signal in this instance is cumulative, the concentration of free donor and acceptor molecules increasing with each cycle of the amplification reaction.

Hybridisation probes are available in a number of forms. Molecular beacons are oligonucleotides that have complementary 5′ and 3′ sequences such that they form hairpin loops. Terminal fluorescent labels are in close proximity for FRET to occur when the hairpin structure is formed. Following hybridisation of molecular beacons to a complementary sequence the fluorescent labels are separated, so FRET does not occur, and this forms the basis of detection. If such as probe is incorporated into the oligonucleotide of the invention however, care must be taken to ensure that the means used to cleave the cleavable region of the probe does not also cleave any duplex structures present within the probe. This would be possible, for example by selecting enzymes which did not affect this region of the probe, for example because it did not contain any sequences recognised by the enzyme, or because it was an RNAse H whilst the probe sequence comprised a DNA only structure.

Pairs of labelled oligonucleotides may also be used in assays. These hybridise in close proximity on a PCR product strand-bringing donor and acceptor molecules together so that FRET can occur. Enhanced FRET is the basis of detection. Variants of this type include using a labelled amplification primer with a single adjacent probe. These pairs of probes or primer and adjacent probe, may for example comprise the first, second, third or additional sequences within the olignonucleotides described above.

Other methods for detecting amplification reactions as they occur are known however, and any of these may be used. Particular examples of such methods are described for example in WO 99/28500, British Patent No. 2,338,301, WO 99/28501 and WO 99/42611.

WO 99/28500 describes a very successful assay for detecting the presence of a target nucleic acid sequence in a sample. In this method, a DNA duplex binding agent and a probe specific for said target sequence, is added to the sample. The probe comprises a reactive molecule able to absorb fluorescence from or donate fluorescent energy to said DNA duplex binding agent. This mixture is then subjected to an amplification reaction in which target nucleic acid is amplified, and conditions are induced either during or after the amplification process in which the probe hybridises to the target sequence. Fluorescence from said sample is monitored.

An alternative form of this assay, which utilises a DNA duplex binding agent which can absorb fluorescent energy from the fluorescent label on the probe but which does not emit visible light, is described in co-pending British Patent Application No. 223563.8.

Any of these primers and probes used in these assays may be incorporated into the oligonucleotides of the invention in order to allow the possibility that these elements can be effectively generated in situ, in the correct stoichiometric amounts, in the assay.

Where it is required that one or more of the functional nucleic acid sequences are required to be added in a stoichiometry other than one to one, then the number of copies of the nucleic acid sequence provided in the oligonucleotide can be adjusted accordingly, each separated by cleavable spacer regions as described above.

Also as described above, the step of forming a cleavable double stranded region within the or each spacer region may be achieved, for instance by adding a short complementary sequence and subjecting the thus formed mixture to conditions under which the complementary sequence will anneal to the spacer region. Alternatively, where the spacer regions includes complementary regions, it may be necessary only to adjust or maintain the temperature of the reaction mixture at an appropriate level so as to ensure that the complementary sequences anneal to each other so that the oligonucleotide achieves a “hairpin” structure.

As mentioned previously, this is particularly useful in the context of amplification reactions, such as the polymerase chain reaction (PCR).

Thus, in a particular embodiment the invention provides a method for conducting an amplification reaction, said method comprising

(i) forming an amplification reaction mixture comprising a sample containing or suspected of containing a target nucleic acid sequence and an oligonucleotide as described above, wherein each of said first and second nucleic acid sequence is a primer or probe sequence,
(ii) subjecting the mixture to conditions under which a cleavable double stranded region is formed within the spacer of the said olignucleotide,
(iii) cleaving said region; and
(iv) amplifying the resultant mixture.

As used herein, the expression “amplification reaction mixture” includes mixtures having at least some of the components necessary for carrying out an amplification reaction. These may include reagents such as polymerase, buffers, magnesium salts etc., as would be well understood in the art. However, if the oligonucleotide comprises for example primers used in the amplification or the detection of the product, these will not be required to be added to the initial amplification reaction mixture as they will be generated in situ in step (ii).

As discussed above, labelled oligonucleotides could be included in the single oligonucleotide to provide one or more probe molecules as well as primers. Alternatively, these may be added to the reaction mixture, either initially or on completion, depending upon factors such as the nature of the assay being conducted and whether it is monitored in real-time, as would be understood in the art.

Step (ii) is carried out in various ways depending upon the sequence and the nature of the oligonucleotide. For instance, where the olignonucleotide contains complementary regions which hybridise together to form a hairpin structure, step (ii) may be carried out simply by subjecting the mixture to conditions under which hybridisation between these regions can occur.

Alternatively however, it may be effected by adding to the reaction mixture a short olignucleotide, and subjecting the mixture to conditions under which this will anneal to the said region.

The way in which step (iii) is carried out will also be dependent to a large extent upon the nature of the olignucleotide being used, as any of the above-described cleavage methods may be suitable. Frequently however, step (iii) will be effected by adding to the reaction mixture an enzyme which is able to cleave double stranded nucleic acid formed by the said two complementary regions, and incubating the mixture for a sufficient period of time and at a sufficient temperature to allow cleavage of the oligonucleotide to proceed, preferably to substantial completion. Preferably the enzyme is an enzyme which is substantially active only at elevated temperatures, for example in excess of 50° C., as this will reduce the artefacts possible during an amplification as discussed above. In particular, these enzymes will show less than 50% of their potential activity, suitably less than 20% and most preferably less than 10% of its activity at temperatures below 50° C.

For example, where the enzyme used to cleave the oligonucleotide is a restriction enzyme such as Taq 1, an incubation temperature of 70° C. may be used.

This may be carried out in an initial high-temperature incubation, which can effectively be used as the first denaturation cycle of the amplification reaction, carried out in step (iv).

Amplification is then conducted in the usual way. Where the first and second nucleic acid sequences are unlabelled first and second primer sequences respectively and no probes have been added to the reaction mixture, the amplification product may then be detected using conventional methods such as gel electrophoresis, followed by visualisation using dyes.

However, where one or more labelled probes have been included in the reaction, other detection techniques such as the TAQMAN™ method, and others described above may be used to detect amplification either during or after completion.

This method of carrying out amplification may be usefully complemented by other “hot start” techniques. In particular for example, the method of WO02/088387, which is incorporated herein in its entirety by reference, may be applied in conjunction with the method described herein. In that case, the activity of the polymerase enzyme present in the amplification reaction mixture is inhibited by the deliberate addition of inhibitory quantities of inorganic pyrophosphate such as tetrasodium pyrophosphate (Na₂P₂O₇).

Further addition of a pyrophosphatase enzyme, in particular one that is substantially active only at elevated temperatures as described in WO02/088387 such as thermostable Ppase obtainable from Sulfolbus acidicaldarius, Thermococcus litoralis or, in particular Aeropyrum pernix, will result in the digestion of the inhibiting pyrophosphate, but only when the amplification reaction mixture is ready and suitably at elevated temperature. The method acts by inhibiting polymerase activity. However, formation of primer or probe artefacts such as primer-dimers, which may interfere with the amplification reaction may still be formed in the reaction mixture prior to amplification.

This technique can therefore be usefully combined with the method of described herein. By combining primers and probes into a single oligonucleotide, the possibility of non-specific binding therebetween is reduced. In a particular embodiment, individual primers and probes are released only on cleavage of the spacer region, and as explained above, this may be controlled so that they become available only as the amplification reaction is about to begin, for example by adding a restriction endonuclease at the required point in time, or by utilising an restriction endonuclease which becomes substantially active only once a certain elevated temperature is reached.

In particular, amplification reaction mixtures can be formed using the single oligonucleotides described herein, which include amplification primer and/or probe sequences. In addition, pyrophosphate and pyrophosphatase enzymes are added 2006/082402 PCT/GB2006/000350 as described in WO02/088387, so as to inhibit the amplification reaction until the desired temperature is reached. By combining these two methods, artefacts caused by mis-priming events as well as by undesired polymerase activity are both reduced and therefore, the amplification reaction can proceed effectively.

The invention will now be particularly described by way of example with reference to the accompanying diagrammatic drawings in which:

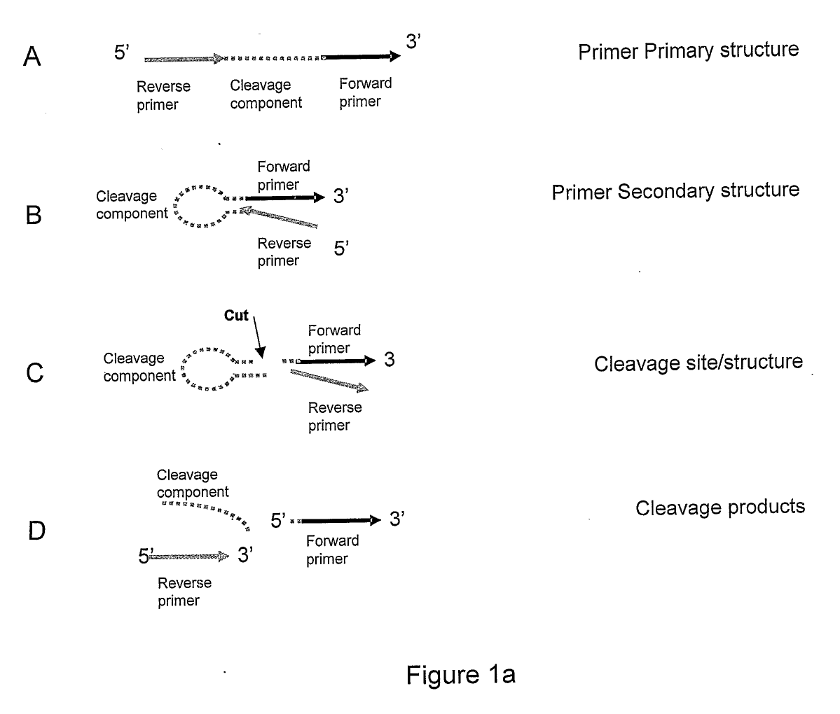

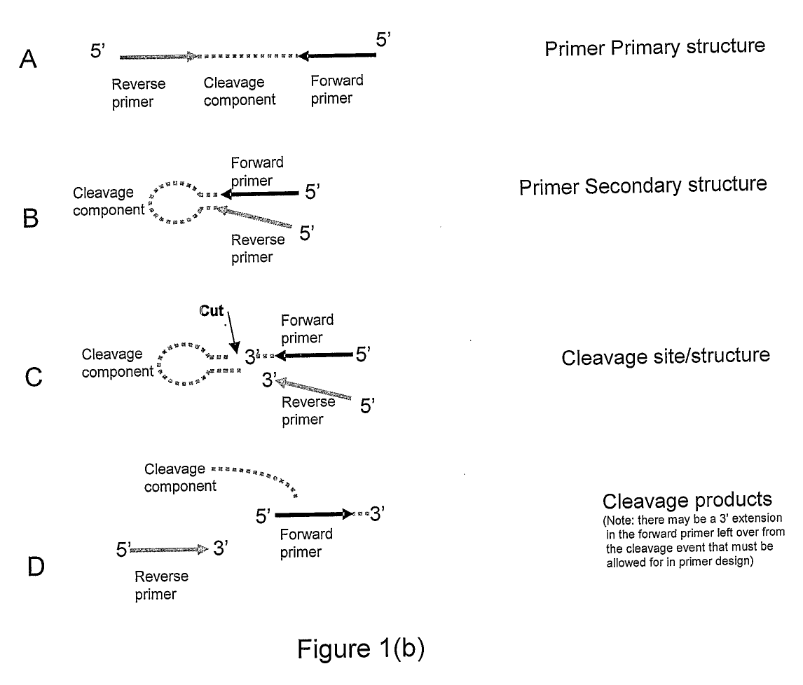

FIG. 1 (a) shows the structure and cleavage of a olignucleotide of an embodiment of the invention, which comprises 5′-3′ double primers, and FIG. 1(b) shows the structure and cleavage of an alternative olignucleotide of the invention, which comprises “back-to-back” primers (5′-3′ . . . 3′-5′ structure);

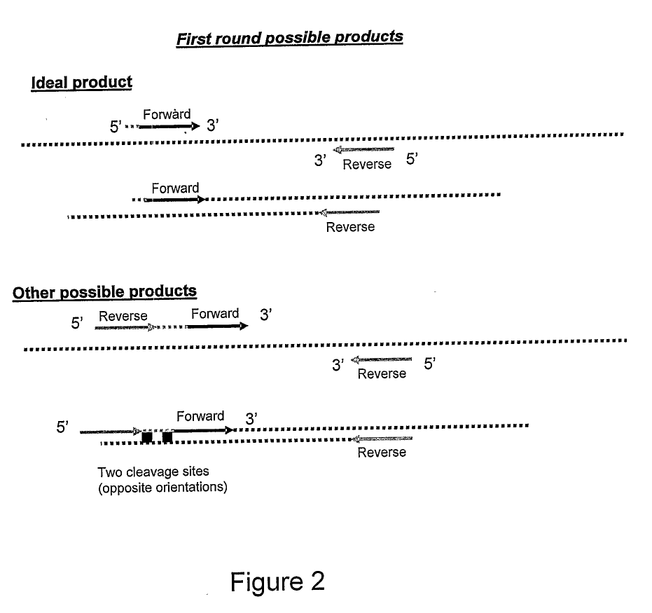

FIG. 2 shows possible specific primer extension products, obtained using oligonucleotides forming an embodiment of the invention; and

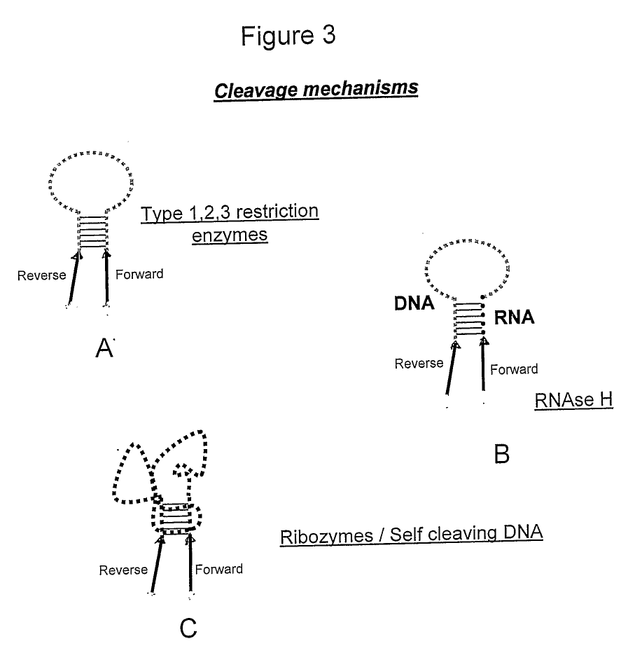

FIG. 3 illustrates various cleavage mechanisms which may be utilised in oligonucleotides of some embodiments of the invention.

In the embodiment of FIG. 1(a), the oligonucleotide comprises a reverse primer sequence at the 5′ end thereof (dark grey), and a forward primer sequence (black) at the 3′ end. These are joined together by way of a spacer region capable of acting as a cleavage component (light grey) FIG. 1(a)(A). This may form a cleavage component by forming, when hybridised to its complementary strand, a sequence recognised by a restriction endonuclease, in which case, addition of a short oligonucleotide comprising the complementary strand would complete the cleavage component.

Preferably however, the spacer region includes two complementary regions which allow the oligonucleotide to adopt a secondary structure in which these regions are hybridised together, so that a “hairpin” structure is formed FIG. 1(a)(B).

The duplex formed by the complementary regions provides a cleavage site or structure (shaded in FIG. 1(a)(C), which is recognised, for example by a particular restriction endonuclease. Incubation of the oligonucleotide with this restriction endonuclease will therefore result in cleavage of the oligonucleotide to form a forward and a reverse primer, and a residual fragment from the spacer region. Although an amount of the spacer region remains attached to the 5′ end of the forward primer in this instance, this will not affect the primer's ability to act as a primer in an amplification reaction such as a PCR.

In the embodiment of FIG. 1(b), the oligonucleotide comprises a reverse primer sequence at the 5′ end thereof (dark grey), and a forward primer sequence (black) at the 3′ end. In this instance however, the forward primer sequence is in the reverse orientation, meaning that the both ends of the oligonucleotide are 5′ ends. The two primer sequences are once again, are joined together by way of a spacer region capable of acting as a cleavage component (mid grey) (FIG. 1(b)(A)), but in this case, it further includes a linker allowing for double ended 3′ attachment. The spacer region further includes a label, indicated by a black dot.

As before, as the spacer region includes two complementary regions, the oligonucleotide tends to adopt a secondary structure in which these regions are hybridised together, so that a “hairpin” structure is formed FIG. 1(b)(B). Again, the duplex formed by the complementary regions provides a cleavage site or structure (shaded in FIG. 1(b)(C), which is recognised, for example by a particular restriction endonuclease. Incubation of the oligonucleotide with this restriction endonuclease will therefore result in cleavage of the oligonucleotide to form a forward and a reverse primer, and a residual labelled fragment from the spacer region (FIG. 1(b)(B)). In this case, the labelled fragment can act as a probe in the subsequent assay, as it includes a region which will hybridise to the target template DNA.

FIG. 2 illustrates the use of the products obtained in FIG. 1(a) in a PCR reaction. The forward and reverse primers act in the usual way, by annealing to complementary strands of template DNA, and are extended to form typical first round products.

In the event that the cleavage of the oligonucleotide is not complete however, the oligonucleotide itself may act as a forward primer. In this case, the first round product will comprise the template sequence having the entire olignucleotide at the 5′-end. In subsequent cycles, the complementary strand of this extended product will be produced, generating two potential cleavage sites with opposite orientations. The action of the restriction endonuclease will have the effect of cutting this artefact, reducing it to the correct product, if such is necessary.

Finally, FIG. 3 illustrates schematically the various cleavage structures which can be produced. Where the oligonucleotide is a DNA molecule (FIG. 3A), it can form a conventional hairpin structure which can be cleaved using for example a Type 1, 2 or 3 restriction enzyme as would be understood in the art. It is necessary only to ensure that the sequence contained in the oligononucleotide is recognised by the particular enzyme used, and is not present in the target template DNA.

Where this is difficult, the oligonucleotide may comprise a DNA/RNA copolymer, wherein the RNA forms an element of the cleavage component (FIG. 3B). On cleavage with an RNAse, the RNA section will be completely digested because it is mispaired with DNA, giving rise to a pair of primers.

Finally, FIG. 3C illustrates a self-cleaving oligonucleotide, where the spacer region comprises a ribozyme able to cleave the duplex formed by the complementary regions of the oligonucleotide.

1. A method for adding a first and a second functional nucleic acid sequence to a reaction mixture in a predetermined stoichiometry and/or at a predetermined point in time, said method comprising adding to the reaction mixture an oligonucleotide comprising a first and a second functional nucleic acid sequence separated by a spacer sequence, said spacer sequence comprising a region which, when double stranded, comprises a cleavable region, forming a cleavable double stranded region within the spacer region of said oligonucleotide, and cleaving the double stranded region within said oligonucleotide.

2. A method according to claim 1 wherein the double stranded cleavable region of the oligonucleotide is cleaved using an enzyme.

3. A method according to claim 2 wherein the olignucleotide is a DNA sequence, and wherein the said enzyme is a restriction endonuclease.

4. A method according to claim 2 wherein the said double stranded region of the spacer sequence of the oligonucleotide comprises an RNA strand and a DNA strand, and wherein said enzyme is an RNAseH or enzyme having RNAseH activity.

5. A method according to claim 1 wherein one of the functional nucleic acid sequences is orientated 5′-3′, and the other functional nucleic acid sequence is orientated 3′-5′ within the oligonucleotide, and these are arranged so that both ends of the oligonucleotide are 5′ ends.

6. A method according to claim 1 wherein the spacer sequence of the oligonucleotide includes two complementary regions which can hybridise together to form a double stranded cleavable region.

7. A method according to claim 6 wherein the said two complementary regions of the spacer sequence of the oligonucleotide are spaced from each other.

8. A method according to claim 7 wherein a third functional nucleic acid sequence is included between said two complementary regions of said oligonucleotide, and wherein said third functional sequence is released on cleavage of the spacer sequence.

9. An method according to claim 1 wherein a third functional nucleic acid is provided between the first and second functional nucleic acids, wherein the spacer sequence includes sufficient regions which, when double stranded, comprise cleavable regions, to allow the oligonucleotide to be cleaved a sufficient number of times to release all the functional sequences therein.

10. A method according to claim 1 wherein one or more functional nucleic acid sequences carries a label.

11. A method for conducting an amplification reaction, said method comprising
(i) forming an amplification reaction mixture comprising a sample containing or suspected of containing a target nucleic acid sequence and an oligonucleotide comprising a first and a second functional nucleic acid sequence separated by a spacer sequence, said spacer sequence comprising a region which, when double stranded, comprises a cleavable region, wherein each of said first and second nucleic acid sequences is a primer or probe sequence,
(ii) subjecting the mixture to conditions under which a cleavable double stranded region is formed within the spacer of the said oligonucleotide,
(iii) cleaving said region; and
(iv) amplifying the resultant mixture.

12. A method according to claim 11 wherein step (iii) is effected by adding to the reaction mixture an enzyme which is able to cleave said double stranded region, and incubating the mixture for a sufficient period of time and at a sufficient temperature to allow cleavage of the oligonucleotide to occur.

13. A method according to claim 12 wherein the olignucleotide is a DNA sequence, and wherein the said enzyme is a restriction endonuclease.

14. A method according to claim 12 wherein the said double stranded region of the spacer sequence of the oligonucleotide comprises an RNA strand and a DNA strand, and wherein said enzyme is an RNAseH or enzyme having RNAseH activity.

15. A method according to claim 11 wherein one of the functional nucleic acid sequences is orientated 5′-3′, and the other functional nucleic acid sequence is orientated 3′-5′ within the oligonucleotide, and these are arranged so that both ends of the oligonucleotide are 5′ ends.

16. A method according to claim 11 wherein the spacer sequence of the oligonucleotide includes two complementary regions which can hybridise together to form a double stranded cleavable region.

17. A method according to claim 16 wherein the said two complementary regions of the spacer sequence of the oligonucleotide are spaced from each other.

18. A method according to claim 17 wherein a third functional nucleic acid sequence is included between said two complementary regions of said oligonucleotide, and wherein said third functional sequence is released on cleavage of the spacer sequence.

19. A method according to claim 11 wherein a third functional nucleic acid is provided between the first and second functional nucleic acids, wherein the spacer sequence includes sufficient regions which, when double stranded, comprise cleavable regions, to allow the oligonucleotide to be cleaved a sufficient number of times to release all the functional sequences therein.

20. A method according to claim 19 wherein the third functional nucleic acid sequence is a probe sequence.

21. A method according to claim 11 wherein one or more functional nucleic acid sequences carries a label.

22. A method according to claim 11 wherein a second oligonucleotide which is shorter than said first oligonucleotide and which is capable of hybridising to said first oligonucleotide is added to the reaction mixture to produce said double stranded cleavable region.

23. A method according to claim 11 wherein the first and second functional nucleic acid sequences are primer sequences.

24. A method according to claim 11 wherein the reaction mixture comprises one or more labelled probes, and the reaction is monitored through the amplification.

25. A method according to claim 12 wherein the said enzyme is substantially active only at elevated temperatures.

26. A method according to claim 11 wherein a pyrophosphate salt is added to the amplification reaction mixture formed in step (i) so as to prevent primer extension taking place, and thereafter, prior to step (iv), said pyrophosphate is digested using a pyrophosphatase enzyme.

27. An oligonucleotide comprising a first and a second functional nucleic acid sequence separated by a spacer sequence, said spacer sequence comprising a region which, when double stranded, comprises a cleavable region.

28. An oligonucleotide according to claim 27 wherein the spacer sequence includes two complementary regions which can hybridise together to form a double stranded cleavable region.

29. An oligonucleotide according to claim 27 wherein the first and second functional nucleic acid sequences are primer sequences.

30. An oligonucleotide according to claim 27 wherein the double stranded cleavable region is cleavable using an enzyme.

31. An oligonucleotide according to claim 30 which is a DNA sequence, and wherein the said enzyme is a restriction endonuclease.

32. An oligonucleotide according to claim 30 wherein the said double stranded region of the spacer sequence comprises an RNA strand and a DNA strand, and wherein said enzyme is an RNAseH or enzyme having RNAseH activity.

33. An oligonucleotide according to claim 27 wherein one of the functional nucleic acid sequences is orientated 5′-3′, and the other functional nucleic acid sequence is orientated 3′-5′ within the oligonucleotide, and these are arranged so that both ends of the oligonucleotide are 5′ ends.

34. An oligonucleotide according to claim 28 wherein the said two complementary regions of the spacer sequence are spaced from each other.

35. An oligonucleotide according to claim 34 wherein a third functional nucleic acid sequence is included between said two complementary regions.

36. An oligonucleotide according to claim 27 wherein a third functional nucleic acid is provided between the first and second functional nucleic acids, wherein the spacer sequence includes sufficient regions which, when double stranded, comprise cleavable regions, to allow the oligonucleotide to be cleaved a sufficient number of times to release all the functional sequences therein.

37. An oligonucleotide according to claim 35 wherein the third functional nucleic acid sequence is a probe sequence.

38. An oligonucleotide according to claim 27 wherein one or more functional nucleic acid sequences carries a label.

39. An oligonucleotide according to claim 38 wherein the labelled functional nucleic acid sequence is a primer.

40. An oligonucleotide according to claim 38 wherein the labelled functional nucleic acid sequence is a probe.

41. A combination of a first oligonucleotide according to claim 27 and a second oligonucleotide which is shorter than said first oligonucleotide and which is capable of hybridising to said first oligonucleotide to produce said double stranded cleavable region.
